(19) United States
(12) Patent Application Publication
(10) Pub. No.: US 2026/0324607 A1
(43) Pub. Date: Oct. 22, 2026

(54) ANIMAL MODEL FOR THE STUDY OF CANCER

(71) Applicants: Centre National de la Recherche Scientifique, PARIS (FR); Sorbonne Université, PARIS (FR); Université Paris Cité, PARIS (FR); École Normale Supérieure, PARIS 5 (FR)

(72) Inventors: David BENSIMON, PARIS (FR); Bertrand DUCOS, GENTILLY (FR); Pierluigi SCERBO, Palaiseau (FR)

(21) Appl. No.: 19/111,304

(22) PCT Filed: Sep. 12, 2023
(86) PCT No.: PCT/EP2023/075020

§ 371 Date: Mar. 12, 2025

(30) Foreign Application Priority Data
Sep. 14, 2022 (EP) 22306350.4

(51) Int. Cl. A01K 67/0275; C12N 15/85

(52) U.S. Cl. CPC A01K 67/0275; C12N 15/8509; A01K 2217/203; A01K 2227/40; A01K 2267/0331; C12N 2015/8572

(57) ABSTRACT

The invention relates to a process for programing a single cell or a group of cells to become malignant, comprising the following steps: a) providing cell(s) with the following features: expressing a dedifferentiation factor, and b) comprising an oncogene whose expression is inducible, and inducing the expression of said oncogene.

### CROSS-REFERENCE TO RELATED APPLICATIONS

The present application is a filing under 35 U.S.C. 371 as the National Stage of International Application No. PCT/EP2023/075020, filed Sep. 12, 2023, entitled “ANIMAL MODEL FOR THE STUDY OF CANCER,” which claims priority to European Application No. 22306350.4 filed with the European Patent Office on Sep. 14, 2022, both of which are incorporated herein by reference in their entirety for all purposes.

### FIELD OF THE INVENTION

The present invention relates to experimental models for studying cancer and in particular the first steps of cancer initiation. More particularly, the invention concerns a process for inducing a single cell or a group of cells to become malignant.

### BACKGROUND OF THE INVENTION

Cancer is considered a rare event resulting from malignant transformation occurring in a single cell. Because this initial event is difficult to observe, it has been difficult to assess the role of the local microenvironment (genetic, cellular, physiological, immunological, etc.) in the initiation and progression of cancer.

In human beings, the initiation of cancer is a rare event considering the number of somatic cells making up a body (~10¹⁴) and the fact that ~10⁹ cells need to be replaced every day. Malignant transformation takes place at the level of individual cells, and only a small subset of these events eventually leads to the outgrowth of tumors.

Specifically, two key unresolved questions of cancer development remain:
(i) the role of mutation(s) in oncogene(s) and/or tumor suppressor gene(s), and
(ii) the role of the cellular intrinsic competence and of the local microenvironment, in the processes of cancer initiation, as well as its progression.

It is commonly accepted that cancer arises through an accumulation of genetic alterations («driver mutations»). Introduction of such genetic alterations in vertebrate models like mouse and zebrafish can recapitulate carcinogenesis. Nevertheless, cancer induction in vivo is often obtained by cooperative mutations, which improve the probability of carcinogenesis, but do not avoid the latency period and the asynchronous carcinogenetic process among sibling.

Studies on naïve tissue samples and cultured cell lines leads to hypothesize that cancers arise from a stem or progenitor cell that regains a “pluripotent-like” state and that fails to respond to internal and environmental cues maintaining tissue homeostasis and cell differentiation. However, no direct experiments have been done at the single cell level in order to shed light on and to track the malignant transformation and initiation of cancers. This is largely due to limited experimental approaches, manipulation and imaging of in vivo models (e.g. mammalian development physiology and size). Furthermore, the lack of non-invasive techniques that unambiguously allow to manipulate, visualize and track cancer initiating cell(s) at single-cell resolution impedes the study of the earliest phases of cell malignant transformation to a carcinogenic cell, and thus fails to give a molecular and cellular identity to the first cancer-initiating cell.

Therefore, both the cell identity and the molecular/cellular mechanism(s) involved in the rise of the cancer-initiating cell during the early events of carcinogenesis are today unknown, as well as the effective impact of gene mutations and the microenvironment leading to the rise of cancers, both in child and adult.

To address those questions in a physiological context, it would be necessary to study the fates of individual cells and their progenies at the very early stages of malignant transformation and cancer progression.

In the model organism zebrafish (danio rerio), a process of optical control of tumor induction has been implemented (Feng et al., 2017). Tumors were initiated by photo-activation of an oncogene (e.g. kRasG12V) in single isolated cells, albeit with a very low probability (~0.5%) in few months.

Zebrafish has the unique advantage of having transparent embryos, which allows to follow precisely its development. Further, 70% of human genes have a counterpart in zebrafish. Currently, zebrafish is widely used as a cancer model, and multiple transgenic lines have been generated that faithfully recapitulated human cancers, including colorectal cancer, melanoma, rhabodomyosarcoma, liver cancer, pancreatic cancer as well as leukemias and brain tumors. The identification of novel cancer signaling pathways and the visualization of pathological processes has been made possible by advances in optical clarity of zebrafish for high resolution imaging, chemical screening using whole animals, and genetic manipulations to generate mutants using genomic editing tools.

However, cancers induced in vivo by modulating oncogenes (e.g. myc, braf) and/or oncosuppressors (e.g. p53, rb, kdm2a, cyclin inhibitors/cdkn) in the whole embryo or in specific tissues generally develop at low rate (~30% of animals) and over an extended period of time (~30-to-60 weeks), both in mice (Donehower et al., 1992) and zebrafish (Kaufman et al., 2016).

Furthermore, global overactivation of canonical oncogenes in zebrafish, like c-KitK642E or BRAFV600E, as well as global genetic depletion of canonical tumor suppressors like tp53, pten or spred1 rarely give rise to cancer in zebrafish within few weeks.

Therefore, it is necessary to develop new in vivo approaches to increase the rate of cancer induction at the single cell level (and not throughout an organism or tissue, or cell-type specific), to reduce the timing of cancer development and to monitor the initial stages of cancer growth.

### SUMMARY OF THE INVENTION

The inventors have addressed these questions by using a process for cancer initiation/programing at single-cell level.

The present invention concerns a process for programing a single cell or a group of cells to become malignant, comprising the following steps:
a) providing cell(s) with the following features:
expressing a dedifferentiation factor, and
comprising an oncogene whose expression is inducible, and
b) inducing the expression of said oncogene.

Specifically, an approach using light to induce the expression of said oncogene, and therefore malignant transformation of a single cell, has been developed. The used animal model is zebrafish larva, but the process can be applied to any animal model adapted for studying cancer.

This process may be implemented in vitro, ex vivo or in vivo.

The present invention also concerns malignant cell(s) obtained by the process as described above.

Another object of the invention is an animal model of cancer comprising at least one malignant cell as described above.

The present invention also related to the use of the malignant cell(s) as described above, or of an animal model as referenced above, for the screening of candidate therapeutic compounds, or disruptors of homeostasis (e.g. environmental pollutants) with potential pro-carcinogenic effects.

Another object of the invention is the use of the malignant cell(s) as described above or of an animal model as referenced above for identifying early biomarkers indicators of the malignancy nature of cells.

### BRIEF DESCRIPTION OF THE FIGURES

FIGS. 1A-1C: Schematic principle and system design of photo-control over specific oncogene expression.

FIG. 1A) A protein of interest (i.e., CRE, black-square) genetically fused with the estrogen receptor (ERT, grey circle) is inactivated by the complex it forms with cytoplasmic chaperones (cc, grey ellipse). This complex can be dissociated by binding of a specific inducer (e.g., cyclofen) to ERT. A caged inducer (caged cyclofen, cCyc, grey square) is chemically synthesized and can be uncaged and activated (Cyclofen, Cyc, grey circle) by illumination at ~375 nm (~750 nm with a two-photon source) and activate protein fused to ERT (i.e., CRE-ERT).

FIG. 1B) This example shows that expression of the oncogene was induced selectively in one muscle fiber cell through localized uncaging of cCyc in a developing zebrafish embryo. Note that embryos were injected with plasmid (actin: loxP-Eos-STOP-loxP-k-RasG12V-T2A-CFP) in a Tg(ubi: Cre-ERT) zebrafish line. EosFP is expressed mosaically, but only the light-activated cell expressed CFP (red arrow denoted).

FIG. 1C) A recently developed zebrafish transgenic line (Tg(actin: loxP-Eos-STOP-loxP-k-RasG12V-T2A-H2B-mTFP; ubi: Cre-ERT)) show ubiquitous expression of EosFP in untreated embryos (3dpf; control, ctrl), whereas photo-activation leads to Cre-ERT-mediated excision of EosFP and allows expression of kRasG12V gene and nuclear mTFP (fused with H2b) in embryos (3dpf) injected with Ventx-GR mRNA (referred as kRasG12V plus Ventx-GR). Expression of nuclear mTFP in kRasG12V plus Ventx-GR embryos (3dpf).

FIG. 2: Induction of cancer in vivo in zebrafish

KRASG 12V plus Ventx-GR, after photo-activation, reduces survival to 20% in 14 dpf (black squares line), whilst control conditions do not alter zebrafish survival.

FIGS. 3A-3D: De-differentiation factor enhances carcinogenesis in vivo.

FIG. 3A) Efficient activation of kRasG12V in zebrafish larvae overexpressing Ventx-GR. Note that, compared to control (ctrl) larvae (expressing ubiquitous EosFP) only larvae overexpressing kRasG12V plus Ventx-GR develop hyperplasia (arrows) (white arrowhead indicates the eye, arrowed the digestive tract)

FIG. 3B) Lateral views (right and left) of zebrafish larvae (8dpf) showing hyperplasic tissues (arrows, delimited by dot circle) (white arrowhead indicates the eye). Note the staining of the nuclei, indicating kRasG12V activation. Percentage of zebrafish larvae (from 6 to 9 dpf) developing hyperplasia in control (non-activated kRasG12V), in activated kRasG12V and in kRasG12V plus Ventx-GR conditions. Note that only kRasG12V plus Ventx-GR efficiently induces hyperplasia within 6-to-9 dpf (82% of larvae).

FIG. 3C) H&E immunostaining of zebrafish larvae (8dpf) section (transversal) in which kRasG12V is expressed with Ventx-GR. Healthy tissues are in the left panel (control, ctrl), tumorigenic and hyperplastic tissues are in the right panel. Only larvae overexpressing kRasG12V plus Ventx-GR develop hyperplasia in arrowed intestine and pancreatic duct, as well as in the brain (cerebellum, grey arrow).

FIG. 3D) Zebrafish larvae (6dpf) were processed for RT-qPCR. Heatmap of larvae over-activating kRasG12V plus Ventx-GR up-regulates (right square) endogenous markers of pluripotency/reprogramming, stemness and epigenetic memory erasure (referred as erasers) and down-regulates endogenous markers (left square) of cell homeostasis (often referred as onco-suppressors) and epigenetic memory maintenance (referred as Writers).

FIGS. 4A-4B: kRasG12V plus Ventx-GR reprogram normal cells to cancer stem cells with migratory and metastatic features.

FIG. 4A) Larvae (6dpf) over-activating kRasG12V plus Ventx-GR were dissociated and isolated cells were transplanted (1 cell per host zebrafish) in host normal zebrafish (2dpf). Note that we used Nacre line, which do not develop melanocytes for a better tracking of transplanted cells. White arrow indicates the eye.

FIG. 4B) Host zebrafish show the transplanted cell (grey-arrow) as soon as 3 h post-transplantation (3hp). After 3 days post transplantation (3dpt), we observed a higher number of exogenous cells in host zebrafish, located in the digestive tract (left panel, grey-arrow), or migrating in the tail (left panel, grey arrow). After 5dpt, cancer-like masses grow in the host zebrafish, indicating that the founder transplanted cell has both migratory, colonizing behaviour, as well as survival growth advantage in the host to form tumors, and thus to re-initialize carcinogenesis. These are specific features of Cancer Stem Cells (CSCs).

FIGS. 5A-5D: kRasG12V plus Ventx-GR induces single-cell malignant transformation and carcinogenesis in vivo.

FIG. 5A) Schematic representation of local co-activation of KRASG12V plus Ventx-GR in a single cell of the brain by laser (375 nm).

FIG. 5B) After 1 dp induction (1dpi), activated cell can be followed by expression of H2b-mTFP and its localization into the nucleus. Otic Vescicle (OV, white arrowhead) and eye (white arrow) are indicated.

FIG. 5C After 5 dpi, the zebrafish brain was colonized by cells (H2b-mTFP positive cells) experienced KRASG12V plus Ventx-GR co-activation (arrows). Note that cancer-like cell masses grow in the brain. Eye (white arrow) are indicated.

FIG. 5D After 5 dpi, H2b-mTFP positive cells migrate far from the site of induction (brain) and colonize ectopic tissues closely located, or within, the digestive tract of zebrafish. These are specific features of metastatic CSCs.

FIGS. 6A-6B: KRASG12V plus NANOG and KRASG12V plus POU5/OCT4 induce hyperplasia.

FIG. 6A) Left: Zebrafish larvae in which expression of KRASG12V was permanently turned on at 1dpf concomittantly with transient (24 h) expression of NANOG (mouse NANOG) develop hyperplasia (white arrowhead, dorsal view at 6dpf). Note that hyperplasic tissues (white arrowhead) are observed in the trunk region where pancreas, liver and digestive tract are located. White arrows indicate the location of the eyes. Right: Magnification of the hyperplasia (dashed white cercle, dashed white arrow) observed in the trunk region of an other similary treated zebrafish larva at 6dpf.

FIG. 6B) Left: Zebrafish larvae in which expression of KRASG12 was permanently turned on at 1dpf concomittantly with transient (24 h) expression of POU 5/OCT4 (Pou5f3.1, a functional homolog of Pou5f1) develop hyperplasia (white arrowhead, dorsal view at 6dpf). Note that hyperplasic tissues (white arrowhead) are observed in the trunk region (dashed white cercle, white arrowhead). White arrow indicates the location of the eye. Right: Magnification of the hyperplasia (dashed white cercle, dashed white arrow) in the trunk/tail region (digestive tract) of an other similarly treated zebrafish larva (6dpf). Scale bars 250 μm.

### DETAILED DESCRIPTION OF EMBODIMENTS OF THE INVENTION

The present invention relates to a process for programing a single cell or a group of cells to become malignant. This process is based on the discovery that a cell expressing both a dedifferentiation factor, and an oncogene, will have higher chances to become malignant than a cell expressing only one of these genes. In this respect, it is important to note that the transient activation of a de-differentiation factor as a method of tumour induction has never been done before.

Advantageously, the expression of at least one of these genes is inducible, therefore allowing a tight control of the spatial and temporal expression of the gene product.

The invention offers various advantages over conventional approaches to tumour induction and studies of anti-tumour agents, as well as homeostasis disruptors with potential pro-carcinogenetic activity.

Indeed, in the process of the invention, the oncogene is activated locally in one (or a few) selected cells in a living organism. On the contrary, current approaches enable the activation of an oncogene on a global scale, in a tissue or in all cells with a given phenotype, but not in a single cell. It is therefore the first time that a process allows to measure the probability of carcinogenesis of a single cell under certain tumour induction conditions.

The coupling of activation of an oncogene with activation, particularly transient induction, of a de-differentiation factor, results in a very high probability of carcinogenesis from a single cell (>80%), in the absence of latency between induction and the appearance of cancer, and in the absence of asynchrony. This paves the way for the development of anti-carcinogenic agents against the early stages of carcinogenesis, or even as prophylactic agents, as well as anti-growth and/or anti-metastatic agents: the aim will be to compare the probability of carcinogenesis (and metastasis) of a cell in a given tissue in the presence or absence of these agents, which is now possible thanks to the process of the invention. Moreover, homeostasis distruptors (e.g. pollutants, contaminants) can be tested for their potential pro-carcinogenetic activity.

It is important to note that another major advantage of the process of the invention lies in the fact that the induction of tumours is closer to the way in which a tumour is supposed to appear in a “normal” physiological context, compared with the techniques of the prior art, namely by clonal development from a mutant cell in a non-mutant tissue of a living organism. In current approaches, all the cells of a given cell type in a given tissue (or the whole tissue, or the whole organism) are mutant and therefore subject to the action of an oncogene (for example by activation of an oncogene such as KRASG12V). In a random and unpredictable way, a tissue cell can then develop a tumour at an indeterminate time. This hinders our understanding of the mechanisms responsible for carcinogenesis and the development of anti-carcinogenic agents, or the effective impact of homeostatic disruptors in promoting or enhancing carcinogenesis. Furthermore, illustrating examples of this process have been performed on zebrafish, a powerful animal model. Zebrafish is a small, transparent and rapidly developing vertebrate which can yield hundreds of eggs on which replicated experiments can be conducted. Many transgenic lines are available that express a photo-activable proteins (light sensitive ionic channels and protein/ligand pairs, etc.) and new ones can be made readily. Consequently, zebrafish is an animal model of choice in protocols that use light to control physiological processes. Moreover, this animal model complies with European Union (EU) Directive 2010/63/EU and the adoption of the 3Rs rule: Replace, Reduce, Refine. In studies (fundamental and translational) of cancer development and in pharmacological tests of anti-cancer molecules (lato sensu), the zebrafish model is an appropriate replacement for other animal models (e.g. mammals including mice, rats etc. . . . in utero development) in accordance with EU directive 2010/63/EU. Analyses are therefore faster, statistically more robust and reproducible, and easier to analyze (external development) than models that do not comply with EU directive 2010/63/EU (mammals), as well as new models (e.g. birds including chickens etc . . . in ovo development) available on the market for pharmacological and/or anticancer tests. The goal of this process of cell programing is to study malignant transformation and further to identify cellular and molecular biomarkers that can predict the rise of cancer in vivo. New predictive biomarkers of malignant transformation can be identified, issued from in vivo analyses in zebrafish.

The present invention concerns a process for programing a single cell or a group of cells to become malignant, comprising the following steps:
a) providing cell(s) with the following features:
expressing a dedifferentiation factor,
comprising an oncogene whose expression is inducible, and
b) inducing the expression of said oncogene.

Unless stated otherwise, the following terms, expressions and phrases as used herein are intended to have the following meanings.

The expression “programing a single cell or a group of cells to become malignant” means that the cell(s) are genetically or epigenetically modified in order to induce phenotypic transformations that will lead to a state of malignity, characterized by cell dedifferentiation (anaplasia), uncontrolled cell proliferation, tumour formation and metastasis capacity.

Malignity nature of a cell, also designated as malignancy, can be established for example by using the “gold standard” histological stain Haematoxylin & Eosin (H&E), well known by the person of the art for cancer detection.

The phrase “inducing the expression” designates the use of a system of expression allowing a controlled expression of the product of the gene, i.e., a system wherein the gene can be unexpressed under certain circumstances, and will not be expressed under other circumstances. For example, the gene may be under the control of an inducible promoter, activated with addition of specific transcription factors or endogenous and (tissue/cell) specific transcription factors Expression of the gene may also be induced by deletion of STOP sequences inserted upstream to the gene, with an inducible recombinase.

The term “dedifferentiation” relates to the process by which cells grow reversely from a partially or terminally differentiated stage to a less differentiated stage within their own lineage. During the dedifferentiation process, development-related gene activity is repressed, and genes that keep the cell in the undifferentiated state are activated. A “dedifferentiation factor”, also designated as “reprograming factor” induces such dedifferentiation. For example, reversine (2-(4-morpholinoanilino)-6-cyclohexylaminopurine) has been identified as a potent dedifferentiation factor. Dedifferentiation factors involved in signaling pathways are in particular the factors involved in the TGF-b signaling pathway, the Notch/Delta pathway, and the Wnt/Beta-catenin and RTK-related pathways.

The term “oncogene” designates a gene that has the potential to cause cancer. In tumour cells, these genes are often mutated and/or expressed at high levels. Products of these genes are usually growth factors, mitogens, receptor tyrosine kinases, serine/threonine-protein kinases such as B-Raf, GTPases such as Ras proteins, or transcription factors such as the product of the myc gene, phosphatases (e.g. PTEN), checkpoint regulators (e.g. TP53).

The present application refers to the appellations of the human genes, but naturally in an animal model, the counterpart gene of said animal (designated as an ortholog) will be expressed in the cell(s) submitted to the process of the invention. The person of the art is able to identify, for each human oncogene, its ortholog/related paralog/functional homolog oncogene in another animal species.

In a specific embodiment of the invention, the oncogene is a gene coding for a serine/threonine kinase. More specifically, the oncogene is BRAF, coding for the BRAF protein involved in transmission of signals into the cells, in particular signals of growth. In a specific embodiment of the invention, the oncogene is a gene coding for a GTPase. GTPases are enzymes with hydrolase activity that bind to the nucleotide guanosine triphosphate (GTP) and hydrolyze it to guanosine diphosphate (GDP). These enzymes are involved in a multitude of fundamental cellular processes. This superfamily includes the class of proteins designated as “small GTPAses”, which itself includes the family of Ras proteins.

In another specific embodiment of the invention, the oncogene is a gene coding for a Ras protein. These proteins designated from “Rat sarcoma virus” are expressed in all animal cell lineages and organs. All Ras protein family members are involved in transmitting signals within cells. Ras activates proteins which activate expression of genes involved in cell growth, differentiation, proliferation and survival. Mutations in Ras genes can lead to the production of aberrantly activated Ras proteins, which can cause unintended and overactive signaling inside the cell, even in the absence of incoming signals or in presence of inactivating proteins. Because these signals result in cell growth and division, overactive Ras signaling can ultimately lead to cancer.

The three Ras genes in humans (HRAS, KRAS and NRAS) are the most common oncogenes in human cancer; mutations that permanently activate Ras are found in 20 to 25% of all human tumors and up to 90% in certain types of cancer. In a specific embodiment of the invention, the oncogene is a KRAS gene, encoding for the KRAS protein. Aberrant KRAS expression, activity and mutations have been found in several cancers, including pancreatic, colorectal, lung, hepatic and brain cancers. As an «oncogenic driver», KRAS likely induces pre-neoplastic lesions that are not sufficient per se for carcinogenesis. In the gene database (GenBank®) edited by the National Center for Biotechnology Information, human KRAS gene is referenced under gene ID: 3845; zebrafish KRAS gene is referenced under gene ID: 445289.

In another specific embodiment of the invention, the oncogene is a HRAS gene, encoding for the HRAS protein.

In another specific embodiment of the invention, the oncogene is a NRAS gene, encoding for the NRAS protein.

In particular, the oncogene is a mutated KRAS gene, for example presenting one of the following point mutations: G12C, G12D or G12V, preferentially a mutated KRAS G12V gene, hereafter designated as kRasG12V.

In another specific embodiment of the invention, the oncogene codes for a transcription factor, in particular is the myc gene.

In another specific embodiment of the invention, the oncogene is a gene coding for a protein of the MAPK pathway. The MAPK (mitogen-activated protein kinase) pathway is a chain of proteins in the cell that communicates a signal from a receptor on the surface of the cell to the DNA in the nucleus of the cell. The signal starts when a signalling molecule binds to the receptor on the cell surface and ends when the DNA in the nucleus expresses a protein and produces some change in the cell, such as cell division. The pathway includes many proteins, such as MAPKs, which communicate by adding phosphate groups to a neighbouring protein (phosphorylating it), thereby acting as an “on” or “off” switch. Therefore, the oncogene may be at least one gene selected among RAF family, such as BRAF, Raf-1 and A-Raf and SRC.

In another specific embodiment of the invention, the oncogene may be a gene coding for mitogenic regulators ligand/receptor types. Indeed, it is known in the state of the art that ligand-activated kinase activity stimulation activates several known downstream pathways, such as MAPK signal transduction cascades. Therefore, the oncogene may be selected among FGF (fibroblast growth factor)/FGFR (fibroblast growth factor receptor) genes, IGF (insulin-like growth factor-1)/IGFR (insulin-like growth factor-1 receptor) genes, EGF (Epidermal Growth Factor)/EGFR (Epidermal Growth Factor receptor) genes, TGFβ/TGFβ-R genes, WNT/beta-catenin and Shh signalling pathways and ERBB receptors genes such as ErbB-2, ErbB-3 and ErbB-4 genes.

### Dedifferentiation Factor

Although reprogramming factors, also designated as dedifferentiation factors, are often associated with stemness and therapeutic resistance in cancer, a few cancer-associated genetic alterations/mutations have been described so far (Rawat et al., 2010; Srivastava et al., 2020).

According to an embodiment of the invention, the at least one dedifferentiation factor is a homeodomain protein, i.e. a transcription factor involved in the regulation of cell potency, differentiation, body patterning and morphogenesis at the early stages of development of organisms, coded by an homeobox gene.

In the sense of the invention, “at least one dedifferentiation factor” significates that one, two or more dedifferentiation factors are expressed in the cell or group of cells submitted to the process of programing to become malignant.

Several dedifferentiation factors are known by the person of the art who will be able to select at least one that will be expressed in the cell(s) submitted to the process.

In particular, this dedifferentiation factor may be any gene belonging to ANTP, HOX, NKX and VENTX/NANOG family, for example VENTX/NANOG. In GenBank®, the human Ventx gene is referenced under gene ID: 27287; the zebrafish nanog gene is referenced under gene ID: 792333.

In another embodiment, the dedifferentiation factor may be any gene belonging to POU family, for example POU5/OCT4. In GenBank®, the human Pou5F1 gene is referenced under gene ID: 5460; the zebrafish Pou5f3 gene is referenced under gene ID: 30333, and is also designated as Oct4.

In another embodiment, the dedifferentiation factor may be any gene belonging to SOX family, for example SOX2 or SOX3. In GenBank®, the human SOX2 gene is referenced under gene ID: 6657; the zebrafish SOX2 gene is referenced under gene ID: 378723. In GenBank®, the human SOX3 gene is referenced under gene ID: 6658; the zebrafish SOX3 gene is referenced under gene ID: 30529.

In another embodiment, the dedifferentiation factor may be any gene belonging to bHLH family, for example to MYC family such as human genes c-my (MYC), I-myc (MYCL), and n-myc (MYCN). In GenBank®, the human MYC gene is referenced under gene ID: 4609; the zebrafish MYC gene is referenced under gene ID: 30686.

In another embodiment, the dedifferentiation factor may be any gene belonging to KRUPPEL/KLF family, for example KLF4. In GenBank®, the human KLF4 gene is referenced under gene ID: 9314. The zebrafish KLF4 gene is referenced under gene ID: 562155.

In another embodiment, the dedifferentiation factor may be any gene belonging to SPALT-LIKE/SALL family, for example SALL4. In GenBank®, the human SALL4 gene is referenced under gene ID: 57167. The zebrafish SALL4 gene is referenced under gene ID: 572527.

In another embodiment, the dedifferentiation factor may be LIN28A. In GenBank®, the human LIN28A gene is referenced under gene ID: 79727. The zebrafish LIN28A gene is referenced under gene ID: 394066.

It was shown that these factors, especially VENTX/NANOG and POU5/OCT4, are abnormally re-activated in metaplastic tissues, preceding «oncogenetic driver(s)» mutation. In particular, VENTX/NANOG have been associated with both pancreatic cancers, as well as with brain cancers.

In an embodiment of the process of the invention, the expression or the activity of the dedifferentiation factor into the cell(s) is inducible. For example, the gene coding for the dedifferentiation factor may be under the control of an inducible promoter.

As is well known by the person of the art, induction of the dedifferentiation factor can be achieved by using an adequate inducible promotor (specific or not), by using a molecule to free the factor from cytoplasmic complexes, or by using optogenetic techniques to induce its activation by light.

In a specific embodiment of the invention, the oncogene is a mutated KRAS gene, for example presenting one of the following point mutations: G12C, G12D or G12V, preferentially a mutated KRAS G12V gene, hereafter designated as kRasG12V, and the dedifferentiation factor is VENTX/NANOG.

### Induction of the Expression of the Oncogene—Step (b) of the Process

As mentioned above, the person of the art knows different ways to induce the expression (i.e., transduction and translation into a protein) of a gene, in the present case of the chosen oncogene, and optionally of the dedifferentiation factor.

In a particular embodiment of the invention, the process of programming cell(s) is characterized in that:
said cell(s) comprise in their genome at least one cassette of expression presenting the following structure:
Promoter—Recombinase recognition site—STOP codon—Recombinase recognition site—oncogene;
the expression of said oncogene is obtained with a recombinase enzyme able to remove, in said cassette, the STOP codon present between the promoter and the oncogene to be expressed.

The recombinase may be for example a CRE recombinase, recognizing the loxP sites, well known by the person of the art. In that case the cassette of expression present in the genome of the cell(s) presents the following structure: Promoter—loxP—STOP codon—loxP—oncogene.

STOP codons are well known by the person of the art. They present the following sequences: TAG, TAA or TGA.

In a specific embodiment, the promoter of the cassette is an ubiquitous promoter.

In a preferred embodiment of the invention, the activity of said recombinase enzyme is inducible, in particular is light-inducible.

In particular, the recombinase enzyme is part of a chimeric protein, also comprising a receptor binding domain that switch in presence of a specific ligand.

In a preferred embodiment, the ligand is a photoactivable ligand.

According to this embodiment, cell(s) submitted to the process has(have) the additional feature of expressing a chimeric protein comprising a recombinase coupled to a switchable receptor binding domain, the recombinase activity depending on the presence of a ligand, preferably of a photoactivable ligand.

A “chimeric protein” designates a protein consisting in different domains originating from different natural or recombinant proteins, covalently linked together with at least one peptide linker.

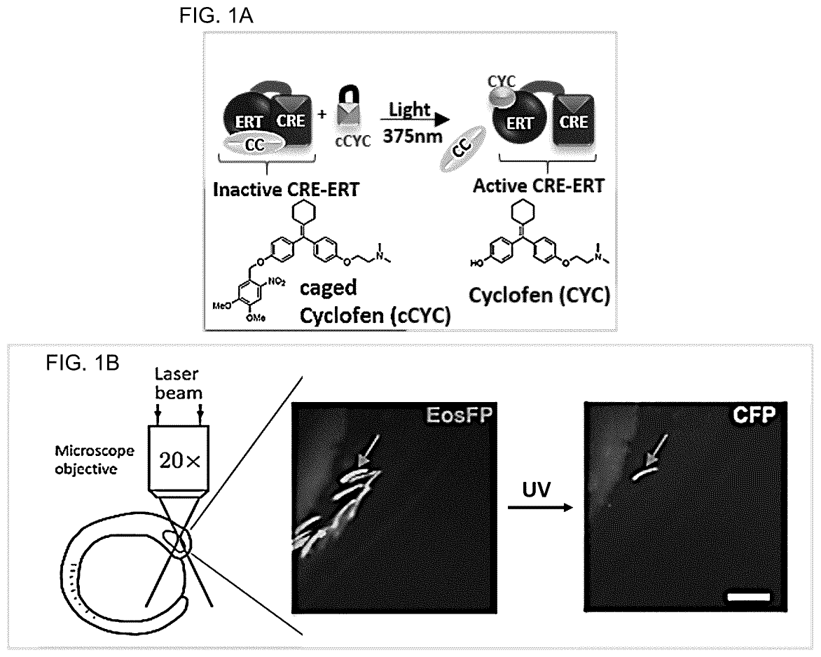

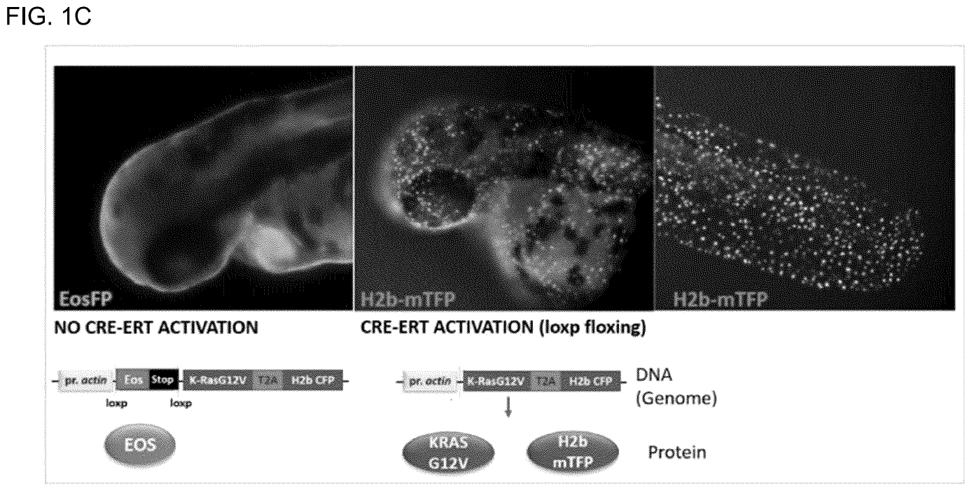

In the present case, the chimeric protein comprises two functional domains (FIG. 1A):
A recombinase, such as CRE, and
A switchable receptor binding domain, defined as a ligand-binding domain from a receptor, able to switch between a “Off” and a “On” position in presence of a specific ligand.

These at least two functional domains are covalently linked with a peptide linker.

In a specific embodiment of the process, the switchable receptor binding domain is the hormone binding domain of the estrogen receptor (ERT) and the photoactivable ligand is a caged cyclofen.

Chimeric proteins comprising the hormone-binding domain of the estrogen receptor (ERT) have been previously reported and are extensively used for inducible nucleus translocation. The tamoxifen inducible system is one of the best-characterized “reversible switch” models. In this system, ERT is used as a regulatory domain: without its ligand tamoxifen, the receptor is maintained under inactive form, in an inhibitory complex, comprising intracellular chaperones such as Hsp90, localized into the cytoplasm. In presence of tamoxifen, the truncated receptor ERT is released from its inhibitory complex and the chimeric protein moves to the nucleus and becomes functional.

Derived switchable domains, such as ERT2 specific of the ligand 4-hydroxytamoxifen (4-OHT or 4-HT), have been engineered.

Another interesting ligand of ERT is the analogue 4-hydroxycyclofen (4-OHC) that has a similar affinity than 4-OHT to ERT, but is easier to synthetize and presents a better photostability.

Recently, photoactivable ligands and corresponding RBD have been developed. These ligands are attached to a photolabile protecting group termed “caging group”. When applied, light removes this caging group and restore the native biological activity of the ligand. In consequence, said ligand exists under two forms designated as “caged” and “uncaged”. Photo-activation at a specific light length allows the “uncaging” of the ligand and therefore its binding to the RBD.

ERT antagonists such as tamoxifen and derivatives can be caged and photo-released to control any chimeric protein comprising an ERT domain.

Photoactivable tamoxifen derivatives have been described, for example, in WO 2013/158268.

Other photoactivable tamoxifen derivatives have been engineered such as caged cyclofen-OH (cCyc), that is currently commercially available as “Actiflash”, distributed by the company IDYLLE (Sinha et al., 2010). Among the caged 4-hydroxycyclofen compounds, at least two versions are proposed: a caged cyclofen-OH that is photo-activated with a wave length of about 365 nm (between 350 and 410 nm), and another one that is photo-activated with a wave length of about 480 nm.

Upon uncaging of cCyc (using UV illumination at ~365 nm or two-photon activation at 750 nm), cyclofen binds to the ERT-receptor and releases the fused protein from its cytoplasmic chaperone. The recombinase can then diffuse into the nucleus and regulate the expression of some targeted genes.

In another specific embodiment of the invention, the switchable receptor binding domain is the hormone binding domain of the glucocorticoid receptor (GR) and the photoactivable ligand is a caged dexamethasone.

Indeed, another well-known switchable RBD is the hormone binding domain of the glucocorticoid receptor (GR), whose natural ligand is dexamethasone.

As described in (Hayashi et al., 2006), two new types of caged ligands, caged 17beta-estradiol and caged dexamethasone, have been synthesized and successfully used in transgenic Arabidopsis plants carrying a steroid hormone-inducible transactivation system.

In a specific implementation of the process according to the invention, step (b) of induction of the expression of the oncogene comprises the two successive sub-steps:
b1) cell(s) expressing a chimeric protein comprising a recombinase coupled to a switchable receptor binding domain are contacted with a photoactivable ligand under its caged form,
b2) cell(s) are irradiated with a wavelength of light for a period of time sufficient to uncage said photoactivable ligand,
wherein said uncaged ligand activates the recombinase which induces the expression of said oncogene.

In a specific embodiment, in step b2), the wavelength of light is monophoton, preferably between 350 nm to 410 nm, or biphoton, preferably at 750 nm. More information on the irradiation conditions may be found in (Sinha et al., 2010).

The process of the invention may be performed in vitro, on cultivated cell(s) or organoids/spheroids or explants, or in vivo, on cell(s) belonging to a living body. In that second case, it is preferable to use biphoton light.

The living body is in particular an animal model of cancer, in particular a mammal, more particularly a rodent, and preferably a mouse (mus musculus).

The living body may also be a vertebrate, for example a fish, and in particular zebrafish (danio rerio).

The process as described above presents the advantage to improve the rate of cancer induction (for the study of its initial events), to reduce the timing of cancer development and therefore to allow the monitoring of the initial stages of tumor growth.

The present invention also concerns a process for programming a single cell or a group of cells to become malignant, comprising the following steps:
a) providing a single cell or a group of cells with the following features:
overexpressing at least one dedifferentiation factor,
comprising in its genome a genetic cassette:
Promoter-loxp-stop-loxp-oncogene, and
expressing a chimeric protein comprising a CRE recombinase coupled to a switchable receptor binding domain, the recombinase activity depending on the presence of a photoactivable ligand,
b1) contacting said cell(s) with said photoactivable ligand under its caged form,
b2) irradiating the cell(s) with a wavelength of light for a period of time sufficient to uncage said photoactivable ligand, that will activate the recombinase, the expression of the oncogene occurring once the recombinase is active.

### Malignant Cells and Animals Such as Obtained

The present invention also concerns the malignant cell(s) obtained by the process as described above.

Naturally, this process also comprises a final step c) of incubating the programed cells, in adequate conditions, for a sufficient period of time allowing the transformation of cell(s) into malignant cell(s).

This period of time is comprised between 5 to 14 days, or even between 2 to 14 days.

Advantageously, the period of incubation is inferior to 14 days, or inferior to 10 days, or even inferior to 7 days. In the zebrafish model, the “incubation period” corresponds to the period from the fertilization designated as “days post fertilization (dpf)”.

Said “adequate conditions” refer to an incubation into an embryo-adapted medium (such as mineral water) at a temperature of 28°+/−1 °C, in the dark or under infra-red light.

The present invention also concerns an animal model of cancer comprising at least one malignant cell as described above, if the process has been implemented on a single cell; or an animal model of cancer comprising a group of malignant cells as described above, if the process has been implemented on a group of cells.

### Uses of the Malignant Cells Such as Obtained

The present invention also relates to the use of the malignant cell(s) as described above, or of an animal model comprising at least one malignant cell as described above, for the screening of candidate therapeutic compounds

Another use of the malignant cells as described above, or of an animal model comprising at least one malignant cell as described above, is for assaying the toxicity of certain compounds.

Another use of the malignant cells as described above, or of an animal model comprising at least one malignant cell as described above, is for testing the effects of compounds that are susceptible to promote cancer development (i.e., for identifying cancerogenic compounds).

The present invention also relates to the use of the malignant cell(s) as described above, or of an animal model comprising at least one malignant cell as described above, for identifying early biomarkers indicators of the malignancy nature of cells. As already presented, the malignant cell(s) obtained with the process of the invention are useful models for the monitoring of the very initial stages of tumor growth.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Though widely used as a vertebrate developmental model since the 1960s, zebrafish has emerged as a cancer model only recently (Ignatius et al., 2016). Approximately 70% of human genes have at least one zebrafish ortholog, including 84% of the genes associated with human diseases. Many genes are highly similar to their human orthologs. For example, the human and zebrafish KRAS proteins are 88.3% identical and the TP 53 genes are 59.2% identical at the protein level. Major residues that are frequently found mutated in human cancers are similarly observed in the zebrafish orthologous proteins.

Therefore, it is to be understood that the illustrated embodiments in zebrafish can be transposed to other species, in particular to other model animals and in particular to mammals, for example mice or rats.

### Example 1. Single Cell Photo-Activation and Carcinogenesis in Zebrafish

An experimental approach has been developed to increase the probability of carcinogenesis in a single cell or a group of cells, by inducing the expression of the kRasG12V oncogene together with a dedifferentiation/reprogramming factor, such as for example Ventx/Nanog gene.

Cancer induction in vivo is often obtained by cooperative mutations, which improve the probability of carcinogenesis, but do not avoid the latency period and the asynchronous carcinogenetic process among sibling. This suggests that additional events are required for the rise of cancer in vivo, both in human and in animal model counterparts, but which sets of alterations are sufficient for malignant transformation remains elusive in most cancers.

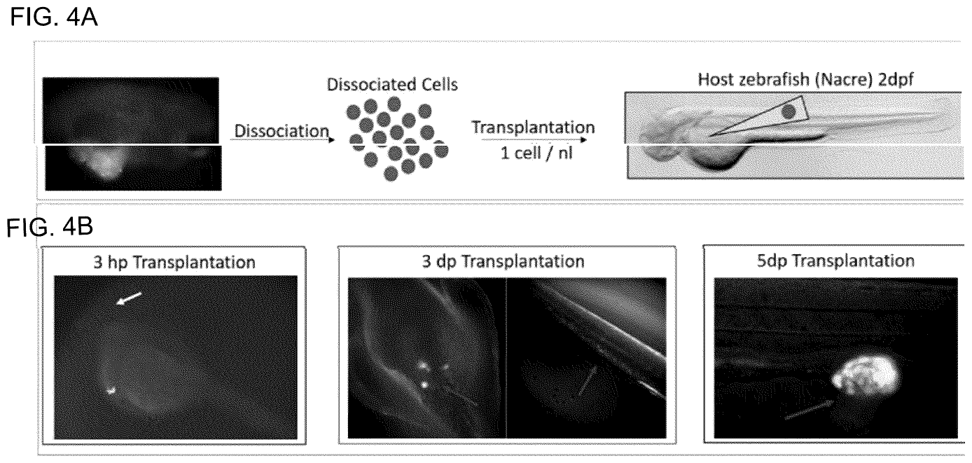

By overexpressing Ventx/Nanog in zebrafish transgenic larvae that carry a photoactivatable (cyclofen-mediated) kRasG12V oncogene, we observed that both global (FIGS. 1A-1C) and single-cell (FIGS. 4A-4B) co-activation of Ventx/Nanog plus kRasG12V can efficiently induce hyperplasic cancer-like structures, tissue outgrowth in vivo, CSCs features in single cells.

More specifically, this approach is developed in zebrafish transgenic lines (such as Tg(actin: loxP-Eos-STOP-loxP-kRasG12V-T2A-H2B-mTFP; ubi: Cre-ERT)) in order to photo-activate a mutated oncogene (kRasG12V) (FIG. 1C).

Embryos ubiquitously expressing Cre-ERT and Eos (a green fluorescent protein) are incubated in caged cyclofen (cCyc) which is photo-activated at 1day post-fertilization (1dpf), releasing the Cre-ERT from its cytoplasmic chaperone complex (FIG. 1A-B).

The Cre recombinase floxes a fluorescent protein (Eos and its stop-codon) and puts the oncogene (kRasG12V) and a T2A mediated marker fluorescent protein (H2B-mTFP) under control of an ubiquitous promoter.

Therefore, KRAS and H2B-mTFP are co-transcribed and translated as separated proteins. Thus H2B-mTFP positive cells are also KRASG12V positive cells.

The larvae can develop hyperplasic tissues albeit with a very low probability (~0.5%) (FIG. 1B).

Such data highlights the necessity to develop new and original approaches in vivo to improve the rate of cancer induction (for the study of its initial events), to reduce the timing of cancer development and to monitor the initial stages of tumor growth.

This approach was improved by using alternative factors for enhancing carcinogenesis in vivo.

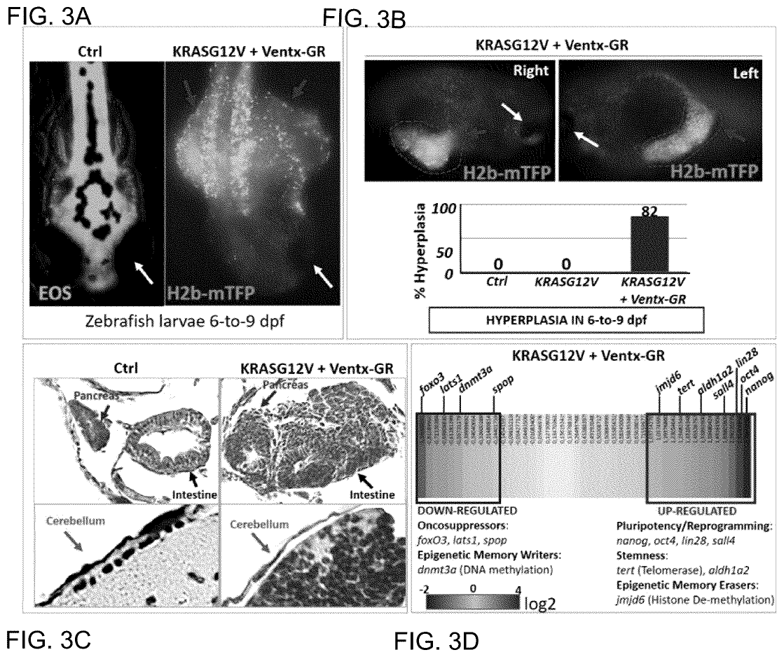

In zebrafish, the process of the invention shows a better efficiency than those presented in the article of (Ablain et al., 2018). In particular, FIGS. 3A-3D of (Ablain et al., 2018) presents various conditions for inducing melanoma in zebrafish, consisting in combined expression of multiple oncogenes (KITK642E, BRAFV600E, NRASQ61R with or without knock-out of classic onco-suppressors tp53, pten, spred1). In comparison with the results of (Ablain et al., 2018), the process of the invention allows to obtain a survival rate that is reduced to 20% after 2 weeks.

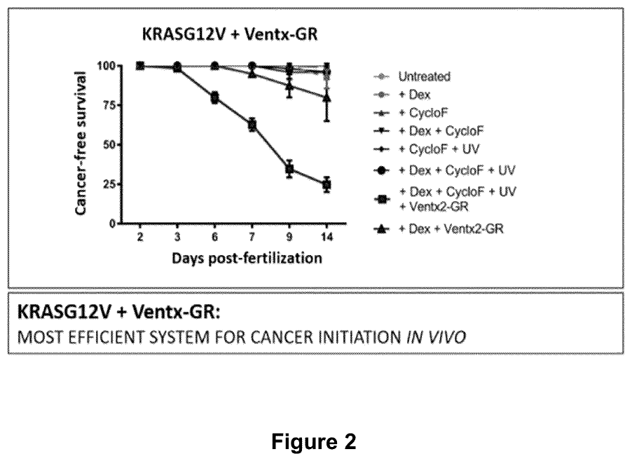

Over-expression of dedifferentiation factors, important for cell pluripotency and reprogramming (i.e., Ventx), together with an oncogene (i.e., kRasG12V) lead to rapid (<two weeks) development of hyperplasic tissue in animals whose survival was ~20% at 14 dpf (FIG. 2, FIGS. 3A-3D).

### Example 2. in Vivo De-Differentiation in Zebrafish at Global and Cell Level

By overexpressing a dexamethasone (DEX) dependent Ventx (Ventx-GR) in zebrafish transgenic larvae that carry a photoactivatable (cyclofen-mediated) kRasG12V oncogene, it was observed that global activation of Ventx plus kRasG12V can efficiently induce hyperplasic cancer-like structures and tissue outgrowth in vivo (FIG. 3A-B).

Such anomalies can arise as soon as 5 up to 14 days post fertilization (dpf) and, importantly, expression of Ventx plus kRasG12V genes can efficiently synergize in inducing hyperplasia (~82%, 42 out of 51 zebrafish larvae) than kRasG12V alone (0%, 0 out of 53 zebrafish larvae) (FIG. 3B).

Accordingly, anatomo-pathological analysis of larval zebrafish, by using “gold standard” histological stain Haematoxylin & Eosin (H&E) for cancer detection, shows that tissues such as the digestive tract (e.g. intestine), as well as the pancreatic duct develop anomalies (FIG. 3C). Brain tissues develop also cancer-like features (FIG. 3C): larval cerebellum (transverse section) shows a strong widespread accumulation of dark staining and further invasion of cancer-like cells into most external-superficial layer of the brain.

A preliminary transcriptional gene screening has been performed in larvae (6dpf) in which Ventx plus kRasG12V are turned on (FIG. 3D). Bona fide markers of induced-nuclear reprogramming have been observed, as well as pluripotency and stemness like pou5/oct4, nanog, lin28, sall4, telomerase (tert), aldh1a2. Further, epigenetic memory erasers (jmjd6) are up-regulated by Ventx plus k-RasG12V.

Conversely, bona fide onco-suppressors (e.g. foxo3, lats1, spop) and “epigenetic memory” regulators (dnmt3a) are down-regulated by the co-expression of Ventx plus k-RasG12V.

This demonstrates that the activation of reprogramming factors, together with a reduction of tumor suppressors and the perturbation of “epigenetic memory”, could drive the initial stages of carcinogenesis in vivo.

### Example 3. Cancer Induction at Single Cell Resolution

It has been shown that transplantation of one cell experiencing co-activation of Ventx plus kRasG12V in a normal non-activated host zebrafish larva was able to proliferate, to migrate and to colonize the host tissues, as well as to give rise to tumor cell masses in the host (FIG. 4A-B).

This suggest that co-activation of Ventx plus kRasG12V can convert normal somatic cells to cancer stem cells (CSCs) with carcinogenic and metastatic features proper to transformed malignant cells, when transplanted in a healthy host organism.

This approach was improved by induction of cancer at single cell resolution.

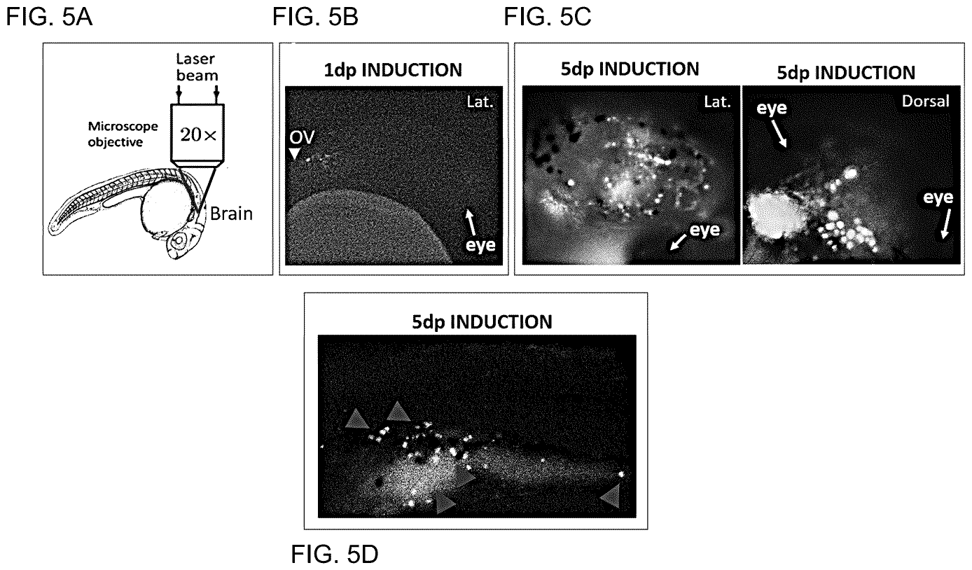

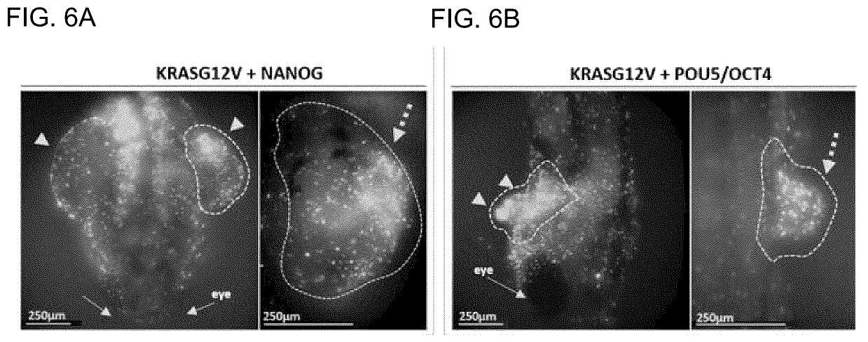

By co-activating Ventx/Nanog plus kRasG12V in a single-cell (or few cells) (FIG. 5A-B) of the zebrafish brain, we observed that carcinogenesis occurs 2 days post-induction, with an efficiency of 70% (FIG. 5C).

This was the first time to our knowledge that a malignant transformation in vivo was followed and tracked, by following a single normal cell undergoing malignant transformation, entering in a proliferative state (FIG. 5C) and further migratory/metastatic behavior (FIG. 5D). Though, we observed that, in 5 days, cancer develops in vivo, without latency or asynchrony among sibling/littermates.

### REFERENCES

WO 2013/158268
Feng Z, Nam S, Hamouri F, Aujard I, Ducos B, Vriz S, Volovitch M, Jullien L, Lin S, Weiss S, Bensimon D. Optical Control of Tumor Induction in the Zebrafish. Sci Rep. 2017 Aug. 23; 7(1): 9195.
Donehower LA, Harvey M, Slagle BL, McArthur MJ, Montgomery CA Jr, Butel JS, Bradley A. Mice deficient for p53 are developmentally normal but susceptible to spontaneous tumours. Nature. 1992 Mar. 19; 356(6366): 215-21.
Kaufman CK, Mosimann C, Fan ZP, Yang S, Thomas AJ, Ablain J, Tan JL, Fogley RD, van Rooijen E, Hagedorn EJ, Ciarlo C, White RM, Matos DA, Puller AC, Santoriello C, Liao EC, Young RA, Zon LI. A zebrafish melanoma model reveals emergence of neural crest identity during melanoma initiation. Science. 2016 Jan. 29; 351(6272): aad2197.
Rawat VP, Arseni N, Ahmed F, Mulaw MA, Thoene S, Heilmeier B, Sadlon T, D'Andrea RJ, Hiddemann W, Bohlander SK, Buske C, Feuring-Buske M. The vent-like homeobox gene VENTX promotes human myeloid differentiation and is highly expressed in acute myeloid leukemia. Proc Natl Acad Sci USA. 2010 Sep. 28; 107(39): 16946-51.

Srivastava Y, Tan DS, Malik V, Weng M, Javed A, Cojocaru V, Wu G, Veerapandian V, Cheung LWT, Jauch R. Cancer-associated missense mutations enhance the pluripotency reprogramming activity of OCT4 and SOX17. FEBS J. 2020 Jan; 287(1): 122-144.
Sinha DK, Neveu P, Gagey N, Aujard I, Benbrahim-Bouzidi C, Le Saux T, Rampon C, Gauron C, Goetz B, Dubruille S, Baaden M, Volovitch M, Bensimon D, Vriz S, Jullien L. Photocontrol of protein activity in cultured cells and zebrafish with one-and two-photon illumination. Chembiochem. 2010 Mar. 22; 11(5): 653-63.

Hayashi K, Hashimoto K, Kusaka N, Yamazoe A, Fukaki H, Tasaka M, Nozaki H. Caged gene-inducer spatially and temporally controls gene expression and plant development in transgenic Arabidopsis plant. Bioorg Med Chem Lett. 2006 May 1; 16(9): 2470-4.
Ignatius MS, Hayes M, Langenau DM. In Vivo Imaging of Cancer in Zebrafish. Adv Exp Med Biol. 2016; 916:219-37.
Ablain J, Xu M, Rothschild H, Jordan RC, Mito JK, Daniels BH, Bell CF, Joseph NM, Wu H, Bastian BC, Zon LI, Yeh I. Human tumor genomics and zebrafish modeling identify SPRED1 loss as a driver of mucosal melanoma. Science. 2018 Nov. 30; 362(6418): 1055-1060.

1. Process for programming a single cell or a group of cells to become malignant, comprising the following steps:
a) providing cell(s) with the following features:
expressing a dedifferentiation factor, and
comprising an oncogene whose expression is inducible, and
b) inducing the expression of said oncogene.

2. Process according to claim 1, wherein the oncogene is selected among a KRAS gene, in particular is kRasG12V gene, HRAS gene, NRAS gene, a RAF gene such as BRAF gene, Raf-1 gene and A-Raf gene, SRC gene, FGF/FGFR genes, IGF/IGFR genes, EGF/EGFR genes, TGFβ/TGFβ-R genes, a WNT/beta-catenin gene, a Shh gene and ERBB receptors genes.

3. Process according to claim 1, wherein said dedifferentiation factor is selected among VENTX/NANOG, POU5/OCT4, a gene belonging to SOX family such as SOX2 or SOX3, a gene belonging to bHLH family, a gene belonging to MYC family such as MYC, a gene belonging to KRUPPEL/KLF family such as KLF4, a gene belonging to SPALT-LIKE/SALL family such as SALL4 and LIN28A.

4. Process according to claim 1, wherein:
i. cell(s) comprise in their genome at least one cassette of expression presenting the following structure:
Promoter—Recombinase recognition site—STOP codon—Recombinase recognition site—oncogene; and
ii. the expression of said oncogene is obtained with a recombinase enzyme able to remove, in said cassette, the STOP codon present between the promoter and the oncogene to be expressed.

5. Process according to claim 4, wherein the activity of said recombinase enzyme is inducible.

6. Process according to claim 5, wherein cell(s) has(have) the additional feature of expressing a chimeric protein comprising a recombinase coupled to a switchable receptor binding domain, the recombinase activity depending on the presence of a ligand, preferably of a photoactivable ligand.

7. Process according to claim 4, wherein:
i) the switchable receptor binding domain is the hormone binding domain of the estrogen receptor (ERT) and the photoactivable ligand is a caged cyclofen, or
ii) the switchable receptor binding domain is the hormone binding domain of the glucocorticoid receptor (GR) and the photoactivable ligand is a caged dexamethasone.

8. Process according to claim 6, wherein step (b) of induction of the expression of said oncogene comprises the following sub-steps:
b1) cell(s) expressing a chimeric protein comprising a recombinase coupled to a switchable receptor binding domain are contacted with a photoactivable ligand under its caged form, and
b2) cell(s) are irradiated with a wavelength of light for a period of time sufficient to uncage said photoactivable ligand,
wherein said uncaged ligand activates the recombinase which induces the expression of said oncogene.

9. Process according to claim 1, wherein the expression or the activity of the dedifferentiation factor is inducible.

10. Process according to claim 1, wherein the process is performed in vitro, on cultivated cell(s) or organoids/spheroids or explants, or in vivo, on cell(s) belonging to a living body.

11. Process according to claim 1, wherein the cell(s) is/are from zebrafish.

12. Malignant cell(s) obtained by the process according to claim 1.

13. Animal model of cancer comprising at least one malignant cell according to claim 12.

14. Use of the malignant cell(s) according to claim 12, for the screening of candidate therapeutic compounds.

15. Use of the malignant cell(s) according to claim 12, for identifying early biomarkers indicators of the malignancy nature of cells.
